(19) United States
(12) Patent Application Publication
(10) Pub. No.: US 2026/0035753 A1
(43) Pub. Date: Feb. 5, 2026

(54) METHODS OF DETERMINING BACTERIA AND MANUFACTURING A FIBROUS WEB AND TOOLS AND USES RELATED THERETO

(71) Applicant: KEMIRA OYJ, Helsinki (FI)

(72) Inventors: Anu JAAKKOLA, Espoo (FI); Jaakko EKMAN, Espoo (FI); Marko KOLARI, Espoo (FI)

(21) Appl. No.: 17/928,702

(22) PCT Filed: Jun. 7, 2021
(86) PCT No.: PCT/FI2021/050420

§ 371 Date: Nov. 30, 2022

(30) Foreign Application Priority Data
Jun. 9, 2020 (FI) 20205594

(51) Int. Cl. C12Q 1/689; D21H 13/08; D21H 21/36

(52) U.S. Cl. CPC C12Q 1/689; D21H 13/08; D21H 21/36; C12Q 2600/16

(57) ABSTRACT

The present invention relates to the fields of determining bacteria and/or manufacturing fibrous webs. Specifically, the invention relates to a method for determining bacteria belonging to the family Thermicanaceae in a cellulose fiber suspension, process water for a production method of a fibrous web, a fibrous web or a machine for producing a fibrous web, and optionally controlling said bacteria, and a method for determining a bacterium belonging to the family Thermicanaceae in a sample. Also, the present invention relates to a method of manufacturing a fibrous web. The present invention further relates to specific primers, primer pairs, probes and kits for determining bacteria belonging to the family Thermicanaceae, and uses thereof. And still, the present invention relates to use of one or more biocides and/or one or more enzyme inhibitors for controlling bacteria belonging to the family Thermicanaceae and a system for controlling said bacteria.

### CROSS REFERENCE TO RELATED APPLICATIONS

This application is a United States National Phase Patent Application of International Patent Application Number PCT/FI2021/050420, filed on Jun. 7, 2021, which claims the benefit of priority to Finnish national application FI 20205594, filed on Jun. 9, 2020.

### SEQUENCE LISTING

A sequence listing confirming to the rules of WIPO Standard ST.25 is hereby incorporated by reference. Said Sequence Listing has been filed as an electronic document via PatentCenter in ASCII formatted text. The electronic document, created on Aug. 26, 2025, is entitled “11354-126US1 Sequence Listing” and is 2,641 bytes in size.

### FIELD OF THE INVENTION

The present invention relates to the fields of determining bacteria and/or manufacturing fibrous webs. Specifically, the invention relates to a method for determining bacteria belonging to the family Thermicanaceae in a cellulose fiber suspension, process water for a production method of a fibrous web, a fibrous web or a machine for producing a fibrous web, and optionally controlling said bacteria, and a method for determining a bacterium belonging to the family Thermicanaceae in a sample. Also, the present invention relates to a method of manufacturing a fibrous web. The present invention further relates to specific primers, primer pairs, probes and kits for determining bacteria belonging to the family Thermicanaceae, and uses thereof. And still, the present invention relates to use of one or more biocides and/or one or more enzyme inhibitors for controlling bacteria belonging to the family Thermicanaceae and a system for controlling said bacteria.

### BACKGROUND OF THE INVENTION

In paper or board mills there can be problems with high microbial growth and poor process conditions. For example, acid production of microbes causes smells in the produced paper or board, and furthermore pH drop can lead to high conductivity in paper or board making process disturbing performance of papermaking chemicals and lowering machine productivity. Some specific microorganisms can cause more problems in very specific processes, process steps or specific process conditions compared to other microorganisms and therefore determinations and optionally control of said specific microorganisms are very important tools for obtaining smooth and cost-effective processes.

For example, publication WO 2016/168430 A1 describes a method of anticipating a microorganism caused problem in a water process system. Said method comprises e.g. measuring the overall microorganism population in at least a portion of the system and measuring the amount of at least one subgroup of microorganism population relative to the overall microorganism population.

But still, there remains a significant unmet need for identification and detection of specific problem causing microorganisms e.g. specific bacteria, which can be used as smart targets of simple, effective, and fast control of paper or board manufacturing processes or any process steps thereof. Indeed, more effective targeted control measures are needed in the field of paper and board industry.

### BRIEF DESCRIPTION

Defects of the prior art including but not limited to lack of knowledge of specific, harmful microorganisms of the paper or board mills and specific tools for determining or controlling said microorganisms can be overcome by the present invention. The inventors of the present disclosure have surprisingly been able to identify specific main spoilage bacteria in paper and board processes or machines. Said specific bacteria can be very abundant, possess problematic metabolic capabilities and/or cause production of acids by fermentation.

The inventors of the present disclosure have now developed fast, effective and specific methods and tools for the detection and enumeration of the specific trouble-making bacteria. Said tools for determining the specific contaminants can be used directly on process samples. The present invention provides tools, such as primers and probes, for determining and/or quantification of bacteria belonging to a specific family, genus or single bacterial species. Indeed, the inventors of the present disclosure have overcome a big challenge to develop a method that detects all the target variants of the bacterial target group but discriminates even the closest relatives outside the target group.

The objects of the present invention, namely methods and tools for monitoring or controlling manufacturing processes of fibrous webs can be achieved by utilizing specific method steps comprising measuring or determining bacteria belonging to the family Thermicanaceae during said manufacturing process or any step thereof.

Surprisingly, bacteria belonging to the family Thermicanaceae can be present in large quantities in paper or board manufacturing processes and result in unwanted process conditions or disturbances in quality of end products. After determining bacteria belonging to the family Thermicanaceae, said bacteria can be controlled by use of one or more biocides and/or one or more enzyme inhibitors.

Furthermore, the inventors of the present disclosure now show that bacteria belonging to the family Thermicanaceae are more common both in percentage (relative abundance) and numbers (absolute abundance) in the same paper or board machine when process conditions get worse (e.g. higher conductivity and/or lower ORP). Therefore, bacteria belonging to the family Thermicanaceae can be used as an indicator of the process health.

The present invention makes it possible to determine and/or reduce bacteria belonging to the family Thermicanaceae in one or more steps when producing a fiber web and furthermore, said bacteria can be reduced in a specific way or to a very specific level when needed.

Indeed, the present invention provides simple and cost-effective industrial scale methods and tools for monitoring and controlling production of fibrous webs. Furthermore, by control methods the amount of used biocidal compositions and/or enzyme inhibitors for treating cellulose fiber suspensions or process water for a production method of a fibrous web can be optimized and thus, an excess use of biocides and/or enzyme inhibitors can be avoided. Improved hygiene and lower chemical load also reduces water consumption and allows closed water circulation processes without increased risk of maintenance break or quality defect of the end product.

In the prior art, bacteria belonging to the family Thermicanaceae have not been monitored and/or controlled in paper or board production processes. An object of the present invention is thus to provide tools and methods for effective and specific monitoring of bacteria belonging to the family Thermicanaceae during a production method of fibrous webs.

The present invention relates to a method of determining bacteria belonging to the family Thermicanaceae in a cellulose fiber suspension, process water for a production method of a fibrous web, a fibrous web or a machine for producing a fibrous web, and controlling said bacteria, wherein the method comprises
determining bacteria belonging to the family Thermicanaceae in a cellulose fiber suspension, process water for a production method of a fibrous web, a fibrous web or a machine for producing a fibrous web, and
controlling bacteria belonging to the family Thermicanaceae by treating the cellulose fiber suspension or process water for a production method of a fibrous web with one or more biocides and/or one or more enzyme inhibitors one or more times.

Also, the present invention relates to a method for determining a bacterium belonging to the family Thermicanaceae in a sample, wherein the method comprises allowing a primer or probe, which is capable of specific hybridization with a polynucleotide of a bacterium belonging to the family Thermicanaceae, to hybridize with said polynucleotide of a sample, and thereby determining the presence or absence of the bacterium or the level of the bacteria belonging to the family Thermicanaceae in said sample.

Also, the present invention relates to a method of manufacturing a fibrous web, such as a paper, board, tissue or the like, wherein the method comprises:
forming an aqueous fiber suspension comprising cellulosic fibers from one or more raw material flows and/or process water,
determining bacteria belonging to the family Thermicanaceae of the aqueous cellulose fiber suspension, raw material flow, process water, a fibrous web, and/or a machine for producing a fibrous web,
optionally controlling bacteria belonging to the family Thermicanaceae by treating the aqueous cellulose fiber suspension or process water with one or more biocides and/or one or more enzyme inhibitors one or more times,
forming the aqueous cellulose fiber suspension into a fibrous web and drying the fibrous web.

Still, the present invention relates to a Thermicanaceae-specific primer or probe capable of specific hybridization with an rRNA gene or an rRNA polynucleotide thereof of a bacterium belonging to the family Thermicanaceae for determining said bacterium in a sample.

Still, the present invention relates to a Thermicanaceae-specific primer pair comprising two primers capable of specific hybridization with an rRNA gene or an rRNA polynucleotide thereof of a bacterium belonging to the family Thermicanaceae for determining said bacterium in a sample.

And still, the present invention relates to a Thermicanaceae-specific kit for determining a bacterium belonging to the family Thermicanaceae in a sample, wherein the kit comprises the primer, probe or primer pair of the present invention and optionally reagents for determining said bacterium belonging to the family Thermicanaceae.

Still furthermore, the present invention relates to use of the primer, probe, primer pair or kit of the present invention for determining the presence, absence or level of bacteria belonging to the family Thermicanaceae in a cellulose fiber suspension, process water for a production method of a fibrous web, a fibrous web or a machine for producing a fibrous web.

Still furthermore, the present invention relates to use of one or more biocides and/or one or more enzyme inhibitors for controlling bacteria belonging to the family Thermicanaceae in a cellulose fiber suspension or process water for a production method of a fibrous web.

And still furthermore, the present invention relates to a system for controlling bacteria belonging to the family Thermicanaceae in a cellulose fiber suspension or process water for a production method of a fibrous web, wherein the system comprises one or more biocides and/or one or more enzyme inhibitors, and the primer, probe, primer pair or kit of the present invention.

Other objects, details and advantages of the present invention will become apparent from the following drawings, detailed description and examples.

### BRIEF DESCRIPTION OF THE DRAWINGS

FIGS. 1A-B show quantification of Thermicanus by qPCR assay with the novel specific primers (on y-axis) vs. quantification by next generation sequencing (NGS) (Thermicanus %) multiplied by total prokaryotic cell count (on x-axis) derived from total DNA yield (FIG. A) or from bacterial qPCR (FIG. B). The quantities on all axes are copies of Thermicanus genomes per volume of a paper process sample.

### SEQUENCE LISTING

SEQ ID NO: 1 shows a polynucleotide sequence of the Thermicanus aegyptius 16S rRNA gene, (strain ET-5b) AJ242495.1.

SEQ ID NO: 2 shows a polynucleotide sequence of a forward primer capable of binding an rRNA gene (e.g. as shown in SEQ ID NO: 1) or an rRNA polynucleotide thereof of the bacterium belonging to the genus Thermicanus (e.g. Thermicanus aegyptius).

SEQ ID NO: 3 shows a polynucleotide sequence of a reverse primer capable of binding an rRNA gene (e.g. as shown in SEQ ID NO: 1) or an rRNA polynucleotide thereof of the bacterium belonging to the genus Thermicanus (e.g. Thermicanus aegyptius).

### DETAILED DESCRIPTION

The present invention concerns a method of monitoring or determining bacteria belonging to the family Thermicanaceae in a cellulose fiber suspension, process water for a production method of a fibrous web, a fibrous web or a machine for producing a fibrous web, and optionally controlling said bacteria. Indeed, the inventors of the present disclosure now discovered that bacteria belonging to a poorly known family Thermicanaceae is very abundant in paper and board manufacturing processes and said bacteria can be controlled to obtain more effective processes and even improved end products.

Cellulosic fibers may be virgin fibers obtained by any known pulping process and/or they may be recycled fibers and/or they may originate from broke. For example, the fiber stock may comprise cellulosic fibers obtained by mechanical pulping, chemical pulping, chemithermomechanical pulping or by repulping recycled or recovered fibers. The cellulosic fibers can be refined or unrefined, bleached or unbleached. The cellulosic fibers may be recycled unbleached or bleached kraft pulp fibers, hardwood semi-chemical pulp fibers, grass pulp fibers or any mixtures thereof. In one embodiment of the invention the cellulose fiber suspension comprises recycled fibers or the fibers of the cellulose fiber suspension are recycled fibers. In another embodiment the fiber suspension comprises fibers from broke or the fibers of the suspension are from broke. In one embodiment the cellulose fiber suspension is an aqueous cellulose fiber suspension, e.g. formed from cellulosic or lignocellulosic fibers, optional papermaking additives and water.

The aqueous cellulose fiber suspension may be formed by combining two or more raw material flows (at least one material flow comprising cellulosic fibers from one or different sources) and/or fresh water and/or circulated process water. The aqueous fiber suspension may contain one or several known chemical additives used in pulp and paper making.

As used herein, “machines for producing fibrous webs” include but are not limited to all paper, tissue and board (such as container board, folding boxboard, liquid packaging board, food service board, liner board, fluting, or core board) machines or systems that use any cellulose containing material such as recycled fiber (RCF). For example, machines using RCF as well as broke systems of any paper grade and wet-lap systems are included within said machines. In one embodiment characteristics of these machines include, but are not limited to, that they use material comprising cellulose and starch.

In one embodiment of the method of the present invention the temperature of the cellulose fiber suspension or process water is at least 40° C., 45° C., 50° C., 55° C., 60° C., 65° C., 70° C., 75° C. or 80° C., typically 45-65° C.; or the temperature of a process utilizing the cellulose fiber suspension, process water or the machine for producing a fibrous web is at least 40° C., 45° C., 50° C., 55° C., 60° C., 65° C., 70° C., 75° C. or 80° C., typically 45-65° C.

In the method of the present invention bacteria belonging to the family Thermicanaceae in a cellulose fiber suspension, process water for a production method of a fibrous web, a fibrous web or a machine for producing a fibrous web is determined, and optionally said bacteria is controlled by treating the cellulose fiber suspension or process water with one or more biocides and/or one or more enzyme inhibitors one or more times.

As used herein “a bacteria belonging to the family Thermicanaceae” refers to any bacteria belonging to the family Thermicanaceae, optionally according to Silva v. 138 taxonomy or GTDB Genome taxonomy database Release 04-RS89 (19 Jun. 2019) taxonomy, and/or to any bacteria belonging to the family Bacillales Family X. Incertae Sedis, optionally according to Bergey's Manual of Systematic Bacteriology, 2^{nd} Ed., and Silva v. 132 taxonomy. Indeed, by the present invention it is possible to determine bacteria belonging to the family Thermicanaceae in general or genus within said family (e.g. Thermicanus) or specific bacteria (e.g. Thermicanus aegyptius). In one embodiment the bacteria belong to the genus Thermicanus or is T. aegyptius. In one embodiment bacteria belonging to the Thermicanaceae family or Thermicanus genus are not slime-forming bacteria.

In one embodiment the method of the present invention comprises determining the potential, presence, absence, amount, level or specific genus or species of the bacteria belonging to the family Thermicanaceae. Any method or tools known in the field of chemistry or biotechnology can be utilized for detecting said bacteria. For example, any microbiological method, community fingerprint techniques including DGGE, T-RFLP and LH-PCR, MALDI, MALDI-TOF, mass spectrometry, and/or any measurement of molecules, organic molecules, polynucleotides, polypeptides, antibodies, enzymes, hormones, secretions or activity associated with said bacteria can be utilized in the present invention. Detections or measurements suitable for the present invention can be either directly or indirectly revealing the bacteria. As used herein “indirect” detections or measurements include but are not limited to those revealing potential bacteria belonging to the family Thermicanaceae. For example, when specific molecules, organic molecules, polynucleotides, polypeptides, antibodies, enzymes, hormones, secretions or activity associated with said bacteria are detected from a sample, the presence of said bacteria in said sample can be indirectly determined.

In one embodiment the presence, absence or level of the bacteria is determined by a molecular method, a nucleic acid-based method or by utilizing one or more primers and/or probes. RNA and/or DNA based methods are suitable molecular methods or nucleic acid methods for the present invention and include but are not limited to hybridization methods (e.g. southern or northern blotting, slot/dot blot, colony blot, fluorescence in situ hybridization, microarray), PCR methods (e.g. qPCR, RT-PCR, qRT-PCR, multiplex-PCR, digital PCR, colony PCR), and sequencing methods (e.g. basic cloning and Sanger sequencing methods, next generation sequencing, high-throughput sequencing). Molecular methods or nucleic acid-based methods may also be combined with any other methods known in the field of biotechnology. E.g. in a specific embodiment the method of the present invention may further comprise a microbiological method such as culturing bacteria on a specific growth media.

In one embodiment the method of the present invention comprises allowing a primer or probe, which is capable of hybridizing with the DNA or RNA of the bacteria, to contact or hybridize with the DNA or RNA of the sample or DNA or RNA obtained from the sample, e.g. in stringent hybridization conditions.

The present invention also concerns a method for determining a bacterium belonging to the family Thermicanaceae in a sample, wherein the method comprises allowing a primer or probe, which is capable of specific hybridization with a polynucleotide of a bacterium belonging to the family Thermicanaceae, to hybridize with said polynucleotide of a sample, e.g. in stringent hybridization conditions.

In one embodiment of the present invention, the method for monitoring or determining a bacterium comprises or is a polymerase chain reaction (PCR) or a quantitative PCR (qPCR). Evidently, reverse transcriptase PCR is used for polymerization of RNA polynucleotides (RT-PCR; RT-qPCR)

In one embodiment hybridization of the primers or probes of the present invention or for the method of the present invention is performed under stringent conditions that allow specific binding (i.e. specific hybridization) between a primer or probe and the target nucleotide sequence. Such stringent conditions for hybridization are sequence-dependent and vary depending on environmental parameters.

In a specific embodiment the stringent conditions are very stringent conditions. For example, stringent or very stringent hybridization conditions can comprise a temperature 50-80° C. (such as 55-75° C., 59-70° C. or 59-63° C., e.g. 60° C., 61° C. or 62° C.) at least 10-30 sec (e.g. at least 15 sec), or at least one or more minutes, e.g. one or more times as is the case during an annealing step of a PCR reaction such as a qPCR reaction. In one embodiment a PCR or qPCR master mix e.g. with 2.2 mM Mg²⁺ (50 mM salt) optionally together with SYBR Green can be utilized in a PCR or qPCR reaction.

Specific (e.g. one, two, three or more) identification sequences have very specific chemical characteristics, and thus it has been very challenging for the inventors of the present disclosure to develop tools for detecting bacteria such as those of specific higher taxonomic level i.e. belonging to the family Thermicanaceae or genus Thermicanus.

In one embodiment of the methods, primers, probes, primer pairs or kits of the present invention, one or more primers or probes is/are capable of hybridizing with an rRNA gene or an rRNA polynucleotide thereof of the bacterium belonging to the genus Thermicanus. A ribosomal RNA gene is a polynucleotide (DNA) encoding rRNA, which is a type of non-coding RNA. rRNA is bound to ribosomal proteins to form small and large ribosome subunits for protein synthesis. In one embodiment the rRNA gene or an rRNA polynucleotide thereof is a 16S rRNA gene or an rRNA thereof.

The present invention further concerns a primer or probe, or a primer pair comprising two primers, capable of hybridizing with an rRNA gene or an rRNA polynucleotide thereof of a bacterium belonging to the family Thermicanaceae for determining said bacterium in a sample. A kit of the present invention for determining a bacterium belonging to the family Thermicanaceae in a sample comprises the primer, probe or primer pair of the present invention. In one embodiment the bacteria belong to the genus Thermicanus.

The primer or probe (i.e. a fragment of DNA or RNA, or a chemically modified fragment of DNA or RNA such as locked nucleic acid, LNA) used in the method or tools of the present invention can be of any suitable length, optionally capable of hybridizing in stringent conditions. For example, the primer or probe can have the length of at least 15 nucleotides, e.g. 18-50 nucleotides, 18-40 nucleotides, 18-30 nucleotides, 18-25 nucleotides, 19-24 nucleotides, 20-23 nucleotides, or 20-22 nucleotides. In some embodiments, probes can be at least 50 nt long, e.g. 100-10000 bases long. The chemically modified or unmodified primers or probes can optionally be labelled for detection.

In one embodiment of the method, primer, probe, primer pair or kit of the present invention, one or more primers or probes is/are capable of hybridizing (e.g. capable of specific hybridization) optionally within SEQ ID NO: 1, e.g. within nucleotides 400-1000, 600-900 or 638-873 as numbered in SEQ ID NO: 1. SEQ ID NO: 1 reveals a polynucleotide sequence of the Thermicanus aegyptius 16S rRNA gene.

In one embodiment of the method, primer, probe, primer pair or kit, the primer is for a polymerase chain reaction (PCR) or a quantitative PCR. In one embodiment the primer or probe comprises a sequence presented in SEQ ID NO: 2 or SEQ ID NO: 3, or the primer pair comprises a first primer comprising a sequence presented in SEQ ID NO: 2 and a second primer comprising a sequence presented in SEQ ID NO: 3. In one embodiment the primer, probe or primer pair comprising a sequence presented in SEQ ID NO: 2 and/or SEQ ID NO: 3 is for a specific binding of an rRNA gene or an rRNA polynucleotide thereof. In one embodiment the primers or probes of the present invention e.g. as presented in SEQ ID NO: 2 and/or 3, cover well current Thermicanaceae or Thermicanus sequences, e.g. RCF Thermicanaceae or Thermicanus.

In one embodiment the specific binding or hybridization of a primer or probe refers to a specific binding or hybridization to DNA or RNA (such as an rRNA gene or an rRNA polynucleotide thereof, e.g. a 16S rRNA gene or an rRNA thereof) of a bacterium belonging to the family Thermicanaceae, the genus Thermicanus or a species within Thermicanaceae or Thermicanus (such as T. aegyptius), and therefore not to DNA or RNA of a bacterium belonging to a family other than Thermicanaceae, a genus other than Thermicanus or a species other than within Thermicanaceae or Thermicanus (such as T. aegyptius), respectively. Indeed, in one embodiment the primer or probe is a Thermicanaceae, Thermicanus or T. aegyptius specific primer or probe.

In one embodiment the primer, probe or kit is for any method of the present invention. The method, primer, probe, primer pair or kit of the present invention can be used for determining the presence, absence or level of bacteria belonging to the family Thermicanaceae in a cellulose fiber suspension, process water for a production method of a fibrous web, a fibrous web or a machine for producing a fibrous web.

In one embodiment the method or kit of the present invention comprise primers and/or probes for detecting two, three, four, five or more bacteria belonging to the family Thermicanaceae (e.g. said bacteria may belong to different genera or to the same genus within the family). In a specific embodiment the method or kit of the present invention further comprises one or more further primers and/or probes for detecting bacteria belonging to a family other than Thermicanaceae (including but not limited to e.g. any family within phylum Proteobacteria and/or phylum Deinococcus-Thermus), genus other than Thermicanus and/or species other than Thermicanus aegyptius.

In addition to comprising the primer, probe or primer pair of the present invention, the kit for determining a Thermicanaceae bacterium optionally comprises reagents for determining said bacterium. Suitable reagents include but are not limited to reaction solutions (e.g. solutions for hybridization, PCR or sequencing based reactions or methods), washing solutions, buffers and/or enzymes. In one embodiment additional tools for determining Thermicanaceae bacteria include but are not limited to detection means selected from the group comprising labels, colouring agents, and/or antibodies or antigen binding fragments. Detection mode of the method or kit of the present invention can be any conventional detection mode including but not limited to e.g. colorimetric or fluorescent detection modes.

Optionally the kit or method of the present invention may also comprise use of any suitable statistical methods, tools and/or instructions related thereto.

In one embodiment the kit further comprises instructions for determining Thermicanaceae bacteria. Said instructions may include but are not limited to instructions selected from the group consisting of instructions for carrying out the method for determining Thermicanaceae bacteria, instructions for carrying out a hybridization, PCR or sequencing based method, conditions of the hybridization, PCR or sequencing based reactions, instructions for detecting the products obtained from the hybridization, PCR or sequencing based reactions, and instructions for interpreting the results.

In a specific embodiment, the kit further comprises one or more control samples. One or more control samples can be selected e.g. from samples comprising Thermicanaceae bacteria or any cell components of said bacteria e.g. produced either by cultivation of cells or synthetically, a specific level of Thermicanaceae bacteria or cell components thereof, samples lacking Thermicanaceae bacteria, or any combination thereof.

The present invention enables detection of bacteria belonging to the family Thermicanaceae from any sample including but not limited to process samples e.g. from paper or board mills. The present disclosure surprisingly reveals that bacteria belonging to the family Thermicanaceae can be used as an indicator of process conditions when manufacturing fibrous webs. The presence, absence, level, increase or decrease of said bacteria can be used for estimating the process conditions. In one embodiment of the method of the present invention, the absence, a decreased or low level, or a level below a pre-determined value of said bacteria indicates good process conditions for producing a fibrous web; decreased or low conductivity of the aqueous cellulose fiber suspension; increased or high pH of the aqueous cellulose fiber suspension; increased or high oxidation-reduction potential (ORP) value of the aqueous cellulose fiber suspension; and/or decreased or low degradation of cellulose and/or starch of the (aqueous) cellulose fiber suspension. In one embodiment the presence, an increased or high level, or a level above a pre-determined value of said bacteria indicates poor process conditions for producing a fibrous web; increased or high conductivity of the aqueous cellulose fiber suspension; decreased or low pH of the aqueous cellulose fiber suspension; decreased or low ORP value of the aqueous cellulose fiber suspension; and/or increased or high degradation of cellulose and/or starch of the (aqueous) cellulose fiber suspension. Therefore, in one embodiment of the method, primer, probe, primer pair or kit of the invention, the primer, primer pair, probe or kit is for indicating process conditions for producing a fibrous web, conductivity or pH of the aqueous cellulose fiber suspension, ORP of the aqueous cellulose fiber suspension, and/or degradation of cellulose and/or starch of the (aqueous) cellulose fiber suspension.

A sample used in the method or for the primer, probe, primer pair or kit of present invention for determining bacteria can be any sample, e.g. a solid or liquid sample, for example an aqueous sample, preferably comprising genetic material. The sample can be obtained from any fibrous web, a machine or system for manufacturing the fibrous web or from any method step of manufacturing fibrous webs, paper or board. In one embodiment there is a high starch content in the sample. In one embodiment the sample is a RCF or RCF process sample. In one embodiment the bacteria belonging to the family Thermicanaceae is determined from a sample obtained from the cellulose fiber suspension (e.g. from an intermediate residence entity), process water for a production method of a fibrous web (e.g. white water), the fibrous web, or the machine for producing a fibrous web. In one embodiment the sample for determining bacteria is (from) a cellulose fiber suspension, process water for a production method of a fibrous web, a fibrous web or a method or machine for producing a fibrous web. The sample to be determined can be from the intermediate residence entity.

In some embodiments the present invention concerns fiber web production methods, machines or parts thereof and includes but is not limited to all paper, tissue or board production systems as well as intermediate residence entities (such as storage towers, broke towers, fiber suspension towers) and process water containers. Aqueous cellulose fiber suspension is formed from a number of raw material flows, typically a plurality of raw material flows, such as water flow and various pulp flows comprising cellulosic fibers. Raw material flows are combined together and form the aqueous fiber suspension which is fed to the intermediate residence entity. The bacteria belonging to the family Thermicanaceae of a fiber suspension, process water, fibrous web, or machine for producing a fibrous web can be determined in any step of producing fiber webs. In one embodiment the bacteria belonging to the family Thermicanaceae is determined before an inlet of an intermediate residence entity, in the intermediate residence entity, and/or after an outlet of an intermediate residence entity. Therefore, the measured Thermicanaceae levels e.g. in an intermediate residence entity can be controlled or adjusted to a desired level for example in said intermediate residence entity and/or after an outlet of said intermediate residence entity. The intermediate residence entity may be any pulp, water or broke storage tower or tank or corresponding entity. In one embodiment the Thermicanaceae bacteria is determined before, in or after a pulp storage tower, pulp storage tank, water tank, broke storage tower and/or broke storage tank, or from a sample obtained before, from or after a pulp storage tower, pulp storage tank, water tank, broke storage tower and/or broke storage tank. According to one embodiment of the invention the method or system for manufacturing a fibrous web comprises a plurality of intermediate residence entities, such as pulp, water or broke storage towers or tanks or corresponding entities or any combination thereof, arranged in the series.

The intermediate residence entity may have a delay time of at least one hour, preferably at least two hours, before the formation of the web. Delay time is here understood as an average residence time (e.g. for the water or aqueous cellulose fiber suspension) in the intermediate residence entity. The intermediate residence entity may have a delay time in the range of 1-48 h, 1-24 h, 1-12 h, typically 1-8 h, more typically 2-7 h. In one embodiment the aqueous cellulose fiber suspension to be determined is in or from an intermediate residence entity with a delay time of 1-48 hours, 1-24 hours, 1-12 hours, typically at least 1 hour or 2 hours, e.g. at least 3, 4, 5, 6, 7, 8, 9, 10, or 11 hours. Typically, the consistency of the aqueous cellulose fiber suspension in the intermediate residence entity is at least 2 g/l, typically in the range of 10-150 g/l.

In one embodiment of the invention bacteria belonging to the family Thermicanaceae is determined (e.g. from a sample) and compared to a pre-determined level. In one embodiment the pre-determined level of bacteria is about, more or less than 1×10⁴, 1×10⁵, 1×10⁶, 1×10⁷, 1×10⁸, 1×10⁹, or 1×10¹⁰ bacteria in ml of the fiber suspension, process water or fibrous web; and/or the pre-determined bacteria level is 0-5%, 0-10%, 0-15%, 0-20%, 0-25%, 0-30%, 0-35%, 0-40%, 0-45%, 0-50%, 0-55%, or 0-60% of total bacteria in the fiber suspension, process water, fibrous web or machine for producing a fibrous web.

In one embodiment the method of the present invention comprises controlling bacteria belonging to the family Thermicanaceae by treating the cellulose fiber suspension or process water for a production method of a fibrous web with one or more biocides and/or one or more enzyme inhibitors one or more times.

No adjustment or control of the Thermicanaceae bacteria is necessary if the determined level of said bacteria is for example absent, low or below the pre-determined value. However, adjustments may be done if deemed advantageous or even necessary e.g. on basis of other parameters. Indeed, after determining the Thermicanaceae bacteria (the first determination) said bacteria can be controlled by either maintaining or adjusting (decreasing or increasing) with one or more biocides and/or one or more enzyme inhibitors either one or more times, if needed.

After determining the Thermicanaceae bacteria, said bacteria (e.g. the level, value, % value, absolute value, proportion, or activity) can be maintained, increased or decreased with one or more biocides and/or one or more enzyme inhibitors, if needed, to obtain a desired final Thermicanaceae bacteria level, value or activity. For example, one or more biocides and/or one or more enzyme inhibitors can be used for maintaining an amount, absolute amount or proportion of Thermicanaceae bacteria in a situation, wherein amount or proportion of Thermicanaceae bacteria would increase without said biocide(s) and/or enzyme inhibitor(s). On the other hand, just small amounts of one or more biocides and/or enzyme inhibitors can result in some increase of the Thermicanaceae bacteria. In one embodiment one or more biocides and/or enzyme inhibitors are used for decreasing the level or proportion of Thermicanaceae bacteria. And still, controlling of Thermicanaceae bacteria includes an option not to use one or more biocides and/or enzyme inhibitors when they are not needed. Thermicanaceae bacteria of the biocide and/or enzyme inhibitor treated fiber suspension or process water can be determined one or more times for confirming the desired obtained level, value, proportion or activity of Thermicanaceae bacteria. One or more biocide and/or enzyme inhibitor treatments or treatment steps may be needed for obtaining the desired Thermicanaceae bacteria level, value, proportion or activity. Indeed, the second, third or more and/or final Thermicanaceae bacteria levels, values, proportions or activity can optionally be determined in order to evaluate the effect of the one or more biocide and/or enzyme inhibitor treatments or a need for further treatments. In one embodiment continuous monitoring by determining Thermicanaceae bacteria, e.g. in a continuous or repetitive manner, and optionally process control by dosing of biocides and/or enzyme inhibitors is utilized.

The determined (first, or optional second, third or more, or final) Thermicanaceae bacteria levels, values, proportion or activity of the fiber suspension or process water can be used for controlling the Thermicanaceae bacteria (e.g. levels, values, proportion or activity) in any process step or system e.g. before an inlet of an intermediate residence entity, in the intermediate residence entity, and/or after an outlet of an intermediate residence entity (such as a pulp storage tower and/or broke storage tower), but e.g. before the aqueous fiber suspension exits the headbox or the like and is formed into a web.

In one embodiment the Thermicanaceae bacteria is controlled (e.g. maintained, increased or decreased) by treating the cellulose fiber suspension or process water with one or more biocides and/or enzyme inhibitors one or more times if the determined Thermicanaceae bacteria level, value, proportion or activity is considered too high or having increasing tendency after two or more determinations. In a specific embodiment the Thermicanaceae bacteria is controlled by treating the cellulose fiber suspension or process water with a biocide and/or enzyme inhibitor one or more times if the determined Thermicanacea bacteria is above a pre-determined value. At least one biocide and/or enzyme inhibitor can be applied to the aqueous cellulose fiber suspension, at least one raw material flows and/or process water. For example, one or more biocides and/or enzyme inhibitors (optionally with other chemicals or agents) can be added to the broke system, broke storage tower(s), broke storage tank(s), pulp, pulp storage tower(s), pulp storage tank(s), water entering the pulper or any storage tank(s), one or more water tanks, and/or pipe line before broke or pulp storage tanks. Indeed, the aqueous cellulose fiber suspension can be treated with one or more biocides and/or enzyme inhibitors e.g. in the broke system, broke storage tower(s), broke storage tank(s), pulp, pulp storage tower(s), and/or pulp storage tank(s). The process water can be treated with one or more biocides and/or enzyme inhibitors e.g. when entering the pulper or any storage tank(s), in a water tank, and/or in a pipe line before broke or pulp storage tanks. In one embodiment Thermicanaceae bacteria level or value is altered with one or more biocides and/or enzyme inhibitors optionally together with a further agent or agents. For example, the number, level, proportion or value of Thermicanaceae bacteria can be reduced, or said bacteria can be eliminated. If biocides and/or enzyme inhibitors are not used for controlling Thermicanaceae bacteria, said bacteria can remain or increase during a method of manufacturing fibrous webs.

In one embodiment, if the determined bacteria belonging to the family Thermicanaceae are abundant, e.g. the level of said bacteria is high (e.g. above a pre-determined value) or estimated to increase (e.g. above a pre-determined value) the bacteria can be controlled to a specific level (e.g. below said pre-determined value), e.g. to a level 0-1×10⁴, 0-1×10⁵, 0-1×10⁶, 0-1×10⁷, 0-1×10⁸, 0-1×10⁹, or 0-1×10¹⁰ bacteria in ml of the fiber suspension or process water, and/or to a level 0-5%, 0-6%, 0-7%, 0-8%, 0-9%, 0-10%, 0-15%, 0-20%, 0-25%, 0-30%, 0-35%, 0-40%, 0-45%, 0-50%, 0-55%, or 0-60% of total bacteria in the fiber suspension or process water (bacteria in ml of the fiber suspension or process water). Total bacteria can be measured for the present invention by any suitable method or combination of methods known to a person skilled in the art including but not limited to measuring the sum of aerobically and anaerobically culturable bacteria, a PCR based method (e.g. qPCR) with general bacterial or prokaryotic primers, a sequencing based method (e.g. NGS), estimation of bacterial biomass by quantification of bacterial genomic DNA, or as a percentage of total bacterial rRNA (e.g. 16S rRNA) gene sequences.

In one embodiment of the invention after determining the bacteria belonging to the family Thermicanaceae said bacteria is controlled by decreasing a level of said bacteria at least at least 5%, 10%, 20%, 30%, 40%, 50%, 60%, 70%, 80%, 90% or 100% (bacteria in ml of the cellulose fiber suspension or process water).

One or more biocide and/or enzyme inhibitor treatment steps may be needed for obtaining a desired Thermicanaceae level.

In one embodiment one or more biocides (either alone or together with one or more enzyme inhibitors and/or other chemical or biochemical agents) are used in the method or system of the present invention for controlling Thermicanaceae bacteria or activities thereof.

In one embodiment one or more biocides used in the method or system of the present invention for controlling bacteria or activities thereof are or comprise an oxidizing biocide and/or a non-oxidizing biocide. In one embodiment the biocide or more than one biocides is/are one or more non-oxidizing biocides selected from the group consisting of: 2,2-Dibromo-3-nitrilopropionamide (DBNPA); 2-Bromo-2-nitropropane-1,3-diol (Bronopol); 2-Bromo-2-nitro-propan-1-ol (BNP); 2,2-Dibromo-2-cyano-N-(3-hydroxypropyl)acetamide; 2,2-Dibromomalonamide; 1,2-Dibromo-2,4-dicyanobutane (DCB); Bis(trichloromethyl)sulfone; 2-Bromo-2-nitrostyrene (BNS); Didecyl-dimethylammonium chlorine (DDAC); ADBAC and other quaternary ammonium compounds; 3-Iodopropynyl-N-butylcarbamate (IPBC); Methyl and Dimethylthiocarbamates and their salts; 5-Chloro-2-methyl-4-isothiazolin-3-one (CMIT); 2-Methyl-4-isothiazolin-3-one (MIT) and their mixture; 2-n-Octyl-4-isothiazolin-3-one (OIT); 4,5-Dichloro-2-(n-octyl)-3(2H)-isothiazolone (DCOIT); 4,5-Dichloro-1,2-dithiol-3-one; 1,2-Benzisothiazolin-3-one (BIT); 2-(Thiocyanomethylthio)benzthiazole (TCMBT); 2-Methyl-1,2-benzisothiazolin-3(2H)-one (MBIT); Tetrakis hydroxymethyl phosphonium sulfate (THPS); Tetrahydro-3,5-dimethyl-2H-1,3,5-thiadiazine-2-thione (Dazomet); Methylene bisthiocyanate (MBT); Ortho-phenylphenol (OPP) and its salts; Glutaraldehyde; Ortho-phthaldehyde (OPA); Guanidines and biguanidines; N-dodecylamine or n-dodecylguanidine; dodecylamine salt or dodecylguanidine salt, such as dodecylguanidine hydrochloride; Bis-(3-aminopropyl)dodecylamine; Pyrithiones, such as Zinc pyrithione; Triazines such as Hexahydro-1,3,5-trimethyl-1,3,5-triazine; 3-[(4-Methylphenyl)sulfonyl]-2-propenenitrile; 3-Phenylsulphonyl-2-propenenitrile; 3-[(4-trifluormethylphenyl)sulphonyl]-2-propenenitrile; 3-[(2,4,6-trimethylphenyl)sulphonyl]-2-propenenitrile; 3-(4-methoxyphenyl)sulphonyl-2-propenenitrile; 3-[(4-methylphenyl)sulphonyl]prop-2-enamide; and any of their isomers; and any combination thereof; and/or
the biocide or more than one biocides is/are one or more oxidizing biocides selected from the group consisting of: chlorine; alkali and alkaline earth hypochlorite salts; hypochlorous acid; bromine; alkali and alkaline earth hypobromite salts; hypobromous acid; chlorine dioxide; ozone; hydrogen peroxide; peroxy compounds, such as performic acid, peracetic acid, percarbonate or persulfate; halogenated hydantoins, such as monohalodimethylhydantoins; dihalodimethylhydantoins; perhalogenated hydantoins; monochloramines; monobromamines; dihaloamines; trihaloamines; urea reacted with an oxidant, the oxidant being e.g. alkali and alkaline earth hypochlorite salts or alkali and alkaline earth hypobromite salts; ammonium salts, e.g. ammonium bromide, ammonium sulfate or ammonium carbamate, reacted with an oxidant, the oxidant being preferably alkali and alkaline earth hypochlorite salts or alkali and alkaline earth hypobromite salts; and any combination thereof.

In one embodiment the biocide used in the method or system of the present invention for controlling cellulolytic activities is or comprises an oxidizing biocide and a non-oxidizing biocide. In one embodiment the non-oxidizing biocide comprises one or more biocides selected from the group consisting of: 2,2-Dibromo-3-nitrilopropionamide (DBNPA); 2-Bromo-2-nitropropane-1,3-diol (Bronopol); 5-Chloro-2-methyl-4-isothiazolin-3-one (CMIT), 2-Methyl-4-isothiazolin-3-one (MIT) and their mixture; Glutaraldehyde; dodecylguanidine hydrochloride; 3-[(4-Methylphenyl)sulfonyl]-2-propenenitrile and any of its isomers; and any combination thereof; and the oxidizing biocide is selected from the group consisting of:
performic acid, monochloramine, ammonium salts reacted with hypochlorite, monochlorodimethylhydantoin or monobromodimethyl hydantoin; and any combination thereof.

In an embodiment the biocide used in the method or system of the present invention for controlling cellulolytic activities comprises one oxidizing biocide selected from a list consisting of performic acid, monochloramines, ammonium salts reacted with hypochlorite, monochlorodimethylhydantoin or monobromodimethyl hydantoin and two or more non-oxidizing biocides selected from a list consisting of 2,2-Dibromo-3-nitrilopropionamide (DBNPA); 2-Bromo-2-nitropropane-1,3-diol (Bronopol); 5-Chloro-2-methyl-4-isothiazolin-3-one (CMIT), 2-Methyl-4-isothiazolin-3-one (MIT) and their mixture; Glutaraldehyde; and dodecylguanidine hydrochloride; 3-[(4-Methylphenyl)sulfonyl]-2-propenenitrile and any of its isomers; and any combination thereof.

The amounts of biocides to be used depend e.g. on the type of fiber suspension or process water used, delay times in residence entities, duration of methods for manufacturing fibrous webs, degree of fresh water usage, the type of the biocide(s) and/or the number of biocide treatments. In one embodiment the fiber suspension or process water is treated with one or more biocides. The added biocide concentration can be e.g. about 0.1-1000 ppm, 1-800 ppm, 3-500 ppm, 5-250 ppm, e.g. about 10, 50, 100, 150 or 200 ppm, based on the active compound content of the biocide. As used herein ppm means a weight of an active compound per volume. In one embodiment the added biocide concentration can be e.g. about 0.1-1000 mg/l, 1-800 mg/l, 3-500 mg/l, 5-250 mg/l, e.g. about 10, 50, 100, 150 or 200 mg/l, based on the active ingredient of the biocide.

In one embodiment one or more enzyme inhibitors (either alone or together with one or more biocides and/or other chemical or biochemical agents) are used in the method or system of the present invention for controlling Thermicanaceae bacteria or activities thereof. Such enzyme inhibitors may be used in a combination with oxidizing and/or non-oxidizing biocides. In one embodiment the enzyme inhibitor comprises zinc ions.

In one embodiment the biocide is a monochloramine. In one embodiment said monochloramine is used in combination with another oxidizing biocide and/or one or more non-oxidizing biocide(s). In one embodiment also one or more enzyme inhibitor(s) is used.

In one embodiment, the zinc ions are derived from or the zinc ion source is selected from an inorganic or organic zinc salt. For example, an inorganic zinc salt can be used as it does not introduce carbon to the manufacturing process which would be usable by microbes. In addition, as inorganic salts are not strongly acidic or alkaline, they do not have any direct effect on the pH. Zinc has been shown to be effective in concentrations that are not harmful for the environment. Furthermore, zinc ions are generally regarded as safe even in applications for human consumption (U.S: Food & Drug Administration; GRAS Substances Database (SCOGS)). In addition, zinc is an inexpensive raw material. In one embodiment, the zinc ions are derived from or the zinc ion source is selected from a group consisting of: ZnBr₂, ZnCl₂, ZnF₂, Zn, ZnO, Zn(OH)₂, ZnS, ZnSe, ZnTe, Zn₃N₂, Zn₃P₂, Zn₃As, Zn₃Sb₂, ZnO₂, ZnH₂, ZnCO₃, Zn(NO₃)₂, Zn(ClO₃)₂, ZnSO₄, Zn₃(PO₄)₂, ZnMoO₄, ZnCrO₄, Zn(AsO₂)₂, Zn(AsO₄)₂, Zn(O₂CCH₃)₂, zinc metal, and any combination thereof. In a specific embodiment zinc salts are selected from the group consisting of ZnCl₂, ZnBr₂, and ZnSO₄, and any combination thereof, and other salts having high solubility in aqueous solutions such as process water.

The amounts of zinc ions to be used can depend e.g. on the fiber suspension or process water used, the type of the biocide and/or the type of zinc ions. In one embodiment the fiber suspension or process water is treated with one or more sources of zinc ions. The added zinc ion concentration can be e.g. about 0.1-500 ppm, 1-400 ppm, 3-250 ppm, 5-100 ppm, e.g. about 10, 20, 30, 40, 50, 60, 70, 80 or 90 ppm zinc ions in the aqueous cellulose fiber suspension or process water. In one embodiment the added zinc ion concentration can be e.g. about 0.1-500 mg/l, 1-400 mg/l, 3-250 mg/l, 5-100 mg/l, e.g. about 10, 20, 30, 40, 50, 60, 70, 80 or 90 mg/l zinc ions in the aqueous cellulose fiber suspension or process water to be treated.

In one embodiment the aqueous cellulose fiber suspension or process water is treated with a combination of one or more biocides and one or more enzyme inhibitors (e.g. added zinc ions such as one or more zinc salts) one or more times. The biocide(s) and enzyme inhibitor(s) can be added simultaneously (e.g. as a pre-mix or separate formulations/products) or consecutively to the cellulose fiber suspension or process water; the biocide(s) can be added prior to the addition of the enzyme inhibitor(s); and/or the enzyme inhibitor(s) can be added prior to the addition of the biocide(s). Also, it is possible to add the biocide(s) continuously and the enzyme inhibitor(s) intermittently, or the enzyme inhibitor(s) continuously and the biocide(s) intermittently. If the biocide(s) and enzyme inhibitor(s) are added consecutively, the time between additions of the biocide(s) and enzyme inhibitor(s) can be e.g. 1 second-180 minutes, 1-60 minutes, 5-30 minutes or 10-20 minutes.

In one embodiment the enzyme inhibitor(s) (e.g. zinc ions) and biocide(s) are used in a ratio of about 1:1 to 100:1, typically 1:10 to 100:1, such as 1:20 to 20:1,1:10 to 10:1, 1:5 to 20:1, 1:5 to 5:1, 1:2 to 5:1, or 1:2 to 2:1. The present invention also concerns a method of manufacturing a fibrous web, such as a paper, board, tissue or the like, wherein the method comprises
forming an aqueous fiber suspension comprising cellulosic fibers from one or more raw material flows and/or process water,
determining bacteria belonging to the family Thermicanaceae of the aqueous cellulose fiber suspension, raw material flow, process water, a fibrous web, and/or a machine for producing a fibrous web,
optionally controlling bacteria belonging to the family Thermicanaceae by treating the aqueous cellulose fiber suspension or process water with one or more biocides and/or one or more enzyme inhibitors one or more times,
forming the aqueous cellulose fiber suspension into a fibrous web and drying the fibrous web.

The aqueous fiber suspension can be formed into a fibrous web and dried in any suitable manner (e.g. by heating and/or removing liquid or water by pressing). The temperature during heating can be e.g. at least 100° C., typically at least 110° C., for at least 0.3 min, e.g. at least 0.5 min, sometimes at least 1 min. The temperature during water removal by pressing may vary and can be e.g. at least RT, typically at least 20° C., 25° C., 40° C., 60° C., 80° C. or at least 100° C.

The present invention further concerns use of one or more biocides and/or one or more enzyme inhibitors for controlling bacteria belonging to the family Thermicanaceae in a cellulose fiber suspension or in process water for a production method of a fibrous web. For example, the biocide(s) and/or enzyme inhibitor(s) can be applied to a broke system, broke storage tower(s), broke storage tank(s), pulp, pulp storage tower(s), pulp storage tank(s), water entering the pulper or any storage tank(s), and/or pipe line before broke or pulp storage tanks. For example the biocides and/or enzyme inhibitors can be used as a premix or as separate agents to be administered separately or at the same time.

A system of the present invention for controlling bacteria belonging to the family Thermicanaceae comprises one or more biocides and/or one or more enzyme inhibitors (as one or more premixes or as separate agents), and the primer, probe, primer pair or kit of the present invention.

In one embodiment the system of the present invention further comprises suitable tools, reagents and/or instructions for determining and/or controlling Thermicanaceae bacteria in a cellulose fiber suspension, process water for a production method of a fibrous web, a fibrous web or a machine for producing a fibrous web. Non-limiting examples of suitable tools include e.g. wells, tubes and tools for taking samples. Suitable reagents include but are not limited to reaction solutions (e.g. solutions for hybridization, PCR or sequencing based reactions or methods) washing solutions, buffers and/or enzymes. In one embodiment additional tools for determining Thermicanaceae bacteria include but are not limited to detection means selected from the group comprising labels, colouring agents, and/or antibodies or antigen binding fragments. Detection mode of the system can be any conventional detection mode including but not limited to e.g. colorimetric or fluorescent detection modes.

The system of the present invention for controlling bacteria can comprise instructions for determining said bacteria in a sample. E.g. said instructions may include instructions selected from the group consisting of instructions for controlling bacteria (e.g. when taking samples, when biocide and/or enzyme inhibitor treatments are needed and when not, what kind of biocide and/or enzyme inhibitor treatments are needed (type, concentration, treatment periods, etc.), instructions for carrying out a method for determining specific bacteria, instructions for taking the samples, instructions for interpreting the results, instructions for carrying out statistical analysis, instructions for one or more biocide and/or enzyme inhibitor treatments, and any combination of said instructions. Optionally instructions may comprise pre-determined values or levels of bacteria to be controlled.

As used in the present disclosure “a polynucleotide” refers to any polynucleotide, such as single or double-stranded DNA (e.g. genomic DNA or cDNA) or RNA (e.g. mRNA, rRNA), optionally comprising a nucleic acid sequence encoding a polypeptide in question or a conservative sequence variant thereof. Conservative nucleotide sequence variants (i.e. nucleotide sequence modifications, which do not significantly alter biological properties of the encoded polypeptide) include variants arising from the degeneration of the genetic code and from silent mutations.

As used in the present disclosure, the terms “polypeptide” and “protein” are used interchangeably to refer to polymers of amino acids of any length.

In the present disclosure, the terms “micro-organism” and “microbe” are used interchangeably.

It will be obvious to a person skilled in the art that, as the technology advances, the inventive concept can be implemented in various ways. The invention and its embodiments are not limited to the examples described below but may vary within the scope of the claims.

### EXAMPLES

### Example 1: Thermicanus is a Novel Bacterial Genus at Board Machine Process Samples

A global next generation sequencing (NGS) based survey of prokaryotic communities at board process samples (white water and pulp), taken from machines that use mainly recycled fiber material, revealed a surprising new group, Thermicanus. Presence of this bacterial genus has not been reported in aqueous paper or board process samples before. At some machines, up to half of the total prokaryotic 16S rRNA gene sequences could be classified into bacterial genus Thermicanus. Original survey was done with Illumina MiSeq sequencing using 16S rRNA primers for variable regions V4-V5, but the finding was verified with multiple technical variations: at four different commercial or academic sequencing laboratories, using several different commercial or non-commercial DNA extraction methods, using primers for 16S rRNA gene variable regions V4 and V1-V3, and using lon Torrent PGM sequencing. The novel detection of Thermicanus at board process samples did not depend on the technical details of the microbiome analysis by NGS.

Out of the tens of board machines surveyed, highest Thermicanus abundances were detected in machines with higher temperatures, such as Mill A (55° C., ˜36%), Mill B board machines 1 and 2 (50° C., ˜33% and ˜22%, respectively), Mill C (45° C., ˜37%), Mill D (46° C., ˜6%), Mill E (˜45° C., ˜3% and ˜9% at different samplings) and Mill F (42° C., ˜17%). Thermicanus was not significantly abundant in process samples with temperature<40° C.

### Example 2: Thermicanus Relatively and Absolutely More Abundant in Worse Board Process Conditions

Some of the machines from Example 1 were sampled again. The relative abundance of main bacterial taxa were quantified by NGS based on 16S rRNA gene sequence proportions as described above, and absolute abundances were determined by multiplying these proportions by total bacterial count quantified by general prokaryotic qPCR (see Table 1). Surprisingly, we found higher relative and absolute abundance of Thermicanus in board process samples taken at a time when worse process conditions prevailed at the machine: sampling times with lower pH, lower ORP and/or higher conductivity.

TABLE 1
Relative and absolute abundances of Thermicanus, total bacteria, and other
board process parameters, at two separate samplings of board machines.

| | Machine | | | | | | | | | |
|---|---|---|---|---|---|---|---|---|---|---|
| | Mill B board machine 1 | | | | Mill B board machine 2 | | | | Mill A | |
| | Sample type | | | | | | | | | |
| | Pulp | | Water | | Pulp | | Water | | Water | |
| Sampling time | 2018 | 2019 | 2018 | 2019 | 2018 | 2019 | 2018 | 2019 | 2018 | 2019 |
| ORP (mV) | −140 | −172 | −60 | −95 | 57 | −160 | 103 | −130 | −35 | 118 |
| Conductivity | 2170 | 3250 | 2200 | 4050 | 1575 | 3020 | 1708 | 3030 | | |
| (μS/cm) | | | | | | | | | | |
| pH | 6.46 | 6.43 | 6.40 | 6.23 | 6.55 | 6.23 | 6.87 | 6.48 | 6.53 | 6.50 |
| Total bacteria | 4.9E+09 | 8.8E+09 | 2.4E+09 | 2.4E+09 | 3.6E+09 | 5.5E+09 | 1.8E+09 | 1.2E+09 | 4.7E+09 | 2.4E+09 |
| (qPCR) | | | | | | | | | | |
| Thermicanus, | 33.2 | 51.3 | 32.5 | 40.5 | 21.2 | 41.1 | 22.8 | 34.2 | 35.6 | 28.9 |
| % | | | | | | | | | | |
| Thermicanus, | 1.6E+09 | 4.5E+09 | 7.9E+08 | 9.9E+08 | 7.6E+08 | 2.3E+09 | 4.2E+08 | 4.0E+08 | 1.7E+09 | 6.9E+08 |
| cells/ml | | | | | | | | | | |

### Example 3: Novel DNA-Based Detection and Quantification Method for Thermicanus

Novel, and to our knowledge the first ever, genus-specific qPCR primers were designed for the genus Thermicanus. Specifically, the primers were designed to discriminate between Thermicanus and Geobacillus, another Firmicutes genus to which some Thermicanus sequences have been falsely classified. Both 16S rRNA gene NGS sequences from Examples 1-2 as well as nucleotide database sequences were used. Potential discriminating priming sites were identified and the primers were designed manually. Properties of potential primers (melt temperatures, secondary structures and dimers) were checked with various tools.

The best primer sequences for a qPCR assay specific for Thermicanus were 5′-CTTGAGGCTAGGAGAGGGAAGT-3′ (SEQ ID NO: 2) and 5′-CAGGCGGAGTGCTTATTGTGT-3′ (SEQ ID NO: 3). Allowing maximum 2 mismatches, these primers detect no 16S rRNA gene of other bacterial genera. The length of the PCR product is 236 bp for Thermicanus aegyptius, the only Thermicanus species described thus far. Specific amplification (one peak in qPCR melt curve analysis) was produced from Thermicanus genomic DNA at annealing temperatures 59-63° C. in qPCR master mix with 2.2 mM Mg²⁺ (50 mM salt) and SYBR Green.

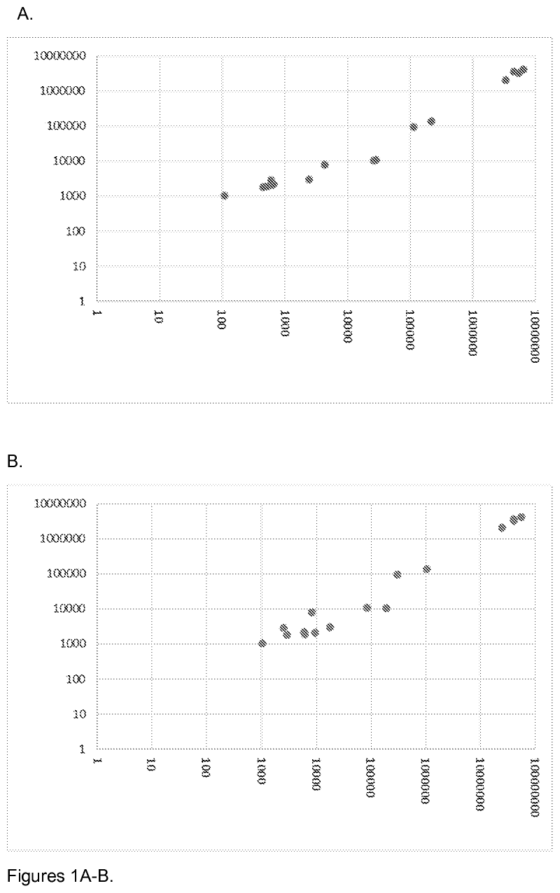

Besides in silico verifications, the validity of the Thermicanus qPCR assay with the novel primers was verified with 16 board process DNA samples from RCF pulp and white water (FIG. 1). Samples were selected based on sequencing results to be taxonomically diverse, include wide range of Thermicanus, and include communities with close relatives of Thermicanus including Geobacillus (Bacillaceae). SYBR Green qPCR detection chemistry was used with hot-start qPCR mastermix on BioRad CFX96 cycler or thermal cycler, using annealing temperature of 61° C. for stringent hybridization. The Pearson correlation between the NGS and qPCR-derived Thermicanus abundance was 0.993, when total bacteria were quantified based on genomic DNA concentration (FIG. 1A), and 0.997, when total bacteria were quantified based on general bacterial qPCR (FIG. 1B). These very high correlations verify the specificity and validity of the novel Thermicanus qPCR primers for the quantification of Thermicanus in board process samples.

### Example 4: Controlling Thermicanaceae Bacteria With One or More Biocides and/or One or More Enzyme Inhibitors

White water from a liner machine using recycled fiber was taken and dry, brown, recycled packaging board was homogenized with the water so that the final consistency of fibers was 2.0%. The pulp was divided into portions and biocides and enzyme inhibitor (zinc) were added into the bottles according to the Table 2. pH of the pulp at the beginning of the test was 7.75 and ORP +175 mV. After this the bottles were incubated at +50° C. for 24 hours. At 24 h pH and ORP were measured and samples for DNA analysis were collected. The amount of Thermicanus was quantified as described in examples 1 and 2.

The results of the test are presented in the Table 2. Biocide and enzyme inhibitor treated samples had clearly higher pH and ORP than control sample. Said biocide and/or enzyme inhibitor treatments also decrease the amount of bacteria belonging to the family Thermicanaceae or genus Thermicanus: The control sample contained 3.2*10⁷ Thermicanus cells/ml, and the treated samples>90% less, 1.2-2.2*10⁶ cells/ml. Thus, in the treated samples both the physical conditions were better and the amount of Thermicanus was lower than in the control sample.

TABLE 2
The results of a microbe control test. After 24 h contact time pH and
ORP were measured and amount of bacteria in the genus Thermicanus
were quantified using NGS sequencing and total bacterial qPCR.

| | | | | | a mixture |
|---|---|---|---|---|---|
| | | | | a mixture | of DBNPA, |
| | | | | of DBNPA, | Bronopol and |
| | | Performic | Mono- | Bronopol and | CMIT/MIT, in |
| | | acid | chloramine | CMIT/MIT, in | total 8 mg/l + |
| | Control | 14 mg/l | 15 mg/l | total 15 mg/l | Zinc 7 mg/l |
| pH | 6.34 | 6.74 | 6.65 | 7.61 | 7.48 |
| ORP (mV) | −548 | +88 | −247 | +84 | +111 |
| Thermicanus, | 3.2*10⁷ | 2.0*10⁶ | 1.2*10⁶ | 2.2*10⁶ | 1.6*10⁶ |
| cells/ml | | | | | |

### Example 5: Controlling Thermicanaceae Bacteria With Monochloramine in Large Scale

A biocide trial on a paper machine using recycled fiber was ran with monochloramine (MCA). Samples from stock storage and cloudy white water were taken 7 months and 1 day before starting the trial and 3 weeks after starting the MCA trial. The amount of Thermicanus was quantified with Thermicanaceae-specific qPCR as described in Example 3. The results of the test are presented in the Table 3. MCA trial decreased Thermicanus densities in stock storage and white water by ≥99%.

TABLE 3
The results of a microbe control trial at a paper machine.
Thermicanus was quantified using specific qPCR.

| | 7 months | 1 day | 3 weeks after |
|---|---|---|---|
| | before | before | start of |
| | trial | trial | MCA trial |
| Thermicanus, | 5.8*10⁸ | 3.9*10⁸ | 3.8*10⁶ |
| stock storage, | | | |
| cells/ml | | | |
| Thermicanus, | 2.7*10⁸ | 2.1*10⁸ | 7.5*10⁵ |
| white water, | | | |
| cells/ml | | | |

1. A method of determining bacteria belonging to the family Thermicanaceae in a cellulose fiber suspension, process water for a production method of a fibrous web, a fibrous web or a machine for producing a fibrous web, and controlling said bacteria, wherein the method comprises:
determining bacteria belonging to the family Thermicanaceae in a cellulose fiber suspension, process water for a production method of a fibrous web, a fibrous web or a machine for producing a fibrous web, and
controlling bacteria belonging to the family Thermicanaceae by treating the cellulose fiber suspension or process water for a production method of a fibrous web with one or more biocides and/or one or more enzyme inhibitors one or more times.

2. The method of claim 1, wherein the bacteria belonging to the family Thermicanaceae is determined from a sample obtained from the cellulose fiber suspension, process water for a production method of a fibrous web, the fibrous web, or the machine for producing a fibrous web.

3. The method of claim 1, wherein presence, absence or level of bacteria belonging to the family Thermicanaceae is determined by a molecular method, a nucleic acid-based method or by utilizing one or more primers and/or probes.

4. The method of claim 1, wherein the bacteria belonging to the family Thermicanaceae is controlled to a level of 0-1×10⁴, 0-1×10⁵, 0-1×10⁶, 0-1×10⁷, 0-1×10⁸, 0-1×10⁹, or 0-1×10¹⁰ bacteria in ml of the fiber suspension or process water, and/or to a level of 0-5%, 0-10%, 0-15%, 0-20%, 0-25%, 0-30%, 0-35%, 0-40%, 0-45%, 0-50%, 0-55% or 0-60% of total bacteria in the fiber suspension or process water; or
the bacteria belonging to the family Thermicanaceae is controlled by decreasing a level of said bacteria by at least 5%.

5. The method of claim 1, wherein the fiber suspension comprises recycled fibers, or the fibers of the fiber suspension are recycled fibers.

6. The method of claim 1, wherein a temperature of the cellulose fiber suspension or process water is at least 40° C.; or a temperature of a process utilizing the cellulose fiber suspension, process water or the machine for producing a fibrous web is at least 40° C.

7. The method of claim 1, wherein at least one of the one or more biocides is a non-oxidizing biocide selected from the group consisting of: 2,2-Dibromo-3-nitrilopropionamide (DBNPA); 2-Bromo-2-nitropropane-1,3-diol (Bronopol); 2-Bromo-2-nitro-propan-1-ol (BNP); 2,2-Dibromo-2-cyano-N-(3-hydroxypropyl)acetamide; 2,2-Dibromomalonamide; 1,2-Dibromo-2,4-dicyanobutane (DCB); Bis(trichloromethyl)sulfone; 2-Bromo-2-nitrostyrene (BNS); Didecyldimethylammonium chlorine (DDAC); ADBAC and other quaternary ammonium compounds; 3-Iodopropynyl-N-butylcarbamate (IPBC); Methyl and Dimethylthiocarbamates and their salts; 5-Chloro-2-methyl-4-isothiazolin-3-one (CMIT); 2-Methyl-4-isothiazolin-3-one (MIT) and their mixture; 2-n-Octyl-4-isothiazolin-3-one (OIT); 4,5-Dichloro-2-(n-octyl)-3(2H)-isothiazolone (DCOIT); 4,5-Dichloro-1,2-dithiol-3-one; 1,2-Benzisothiazolin-3-one (BIT); 2-(Thiocyanomethylthio)benzthiazole (TCMBT); 2-Methyl-1,2-benzisothiazolin-3(2H)-one (MBIT); Tetrakis hydroxymethyl phosphonium sulfate (THPS); Tetrahydro-3,5-dimethyl-2H-1,3,5-thiadiazine-2-thione (Dazomet); Methylene bisthiocyanate (MBT); Ortho-phenylphenol (OPP) and its salts; Glutaraldehyde; Ortho-phthaldehyde (OPA); Guanidines and biguanidines; N-dodecylamine or n-dodecylguanidine; dodecylamine salt or dodecylguanidine salt, such as dodecylguanidine hydrochloride; Bis-(3-aminopropyl)dodecylamine; Pyrithiones, such as Zinc pyrithione; Triazines such as Hexahydro-1,3,5-trimethyl-1,3,5-triazine; 3-[(4-Methylphenyl)sulfonyl]-2-propenenitrile; 3-Phenylsulphonyl-2-propenenitrile; 3-[(4-trifluormethylphenyl)sulphonyl]-2-propenenitrile; 3-[(2,4,6-trimethylphenyl)sulphonyl]-2-propenenitrile; 3-(4-methoxyphenyl)sulphonyl-2-propenenitrile; 3-[(4-methylphenyl)sulphonyl]prop-2-enamide; any of their isomers; and any combination thereof; and/or
at least one of the one or more biocides is an oxidizing biocide selected from the group consisting of: chlorine; alkali and alkaline earth hypochlorite salts; hypochlorous acid; bromine; alkali and alkaline earth hypobromite salts; hypobromous acid; chlorine dioxide; ozone; hydrogen peroxide; peroxy compounds, such as performic acid, peracetic acid, percarbonate or persulfate; halogenated hydantoins, such as monohalodimethylhydantoins; dihalodimethylhydantoins; perhalogenated hydantoins; monochloramines; monobromamines; dihaloamines; trihaloamines; urea reacted with an oxidant, the oxidant being e.g. alkali and alkaline earth hypochlorite salts or alkali and alkaline earth hypobromite salts; ammonium salts, e.g. ammonium bromide, ammonium sulfate or ammonium carbamate, reacted with an oxidant, the oxidant being preferably alkali and alkaline earth hypochlorite salts or alkali and alkaline earth hypobromite salts; and any combination thereof.

8. The method of claim 1, wherein at least one of the one or more enzyme inhibitors comprises zinc ions; or at least one of the one or more enzyme inhibitors comprises zinc ions derived from an inorganic or organic zinc salt.

9. A method for determining a bacterium belonging to the family Thermicanaceae in a sample, wherein the method comprises allowing a primer or probe, which is capable of specific hybridization with a polynucleotide of a bacterium belonging to the family Thermicanaceae, to hybridize with said polynucleotide of a sample, and thereby determining the presence or absence of the bacterium or the level of the bacteria belonging to the family Thermicanaceae in said sample.

10. The method of claim 9, wherein the bacteria belong to the genus Thermicanus.

11. The method of claim 9, wherein the method for determining a bacterium belonging to the family Thermicanaceae in a sample comprises or consists of a polymerase chain reaction (PCR) or a quantitative PCR.

12. The method of claim 9, wherein one or more primers or probes is/are capable of hybridizing with an rRNA gene or an rRNA polynucleotide thereof of the bacterium belonging to the genus Thermicanus, optionally within SEQ ID NO: 1, and/or within nucleotides 400-1000 or 600-900 as numbered in SEQ ID NO: 1.

13. The method of claim 9, wherein the primer or probe comprises a sequence presented in SEQ ID NO: 2 or SEQ ID NO: 3, or a primer pair for determining the bacterium belonging to the family Thermicanaceae comprises a first primer comprising a sequence presented in SEQ ID NO: 2 and a second primer comprising a sequence presented in SEQ ID NO: 3.

14. A method of manufacturing a fibrous web, such as a paper, board, tissue or the like, wherein the method comprises:
forming an aqueous fiber suspension comprising cellulosic fibers from one or more raw material flows and/or process water;
determining bacteria belonging to the family Thermicanaceae of the aqueous cellulose fiber suspension, raw material flow, process water, a fibrous web, and/or a machine for producing a fibrous web;
optionally controlling bacteria belonging to the family Thermicanaceae by treating the aqueous cellulose fiber suspension or process water with one or more biocides and/or one or more enzyme inhibitors one or more times;
forming the aqueous cellulose fiber suspension into a fibrous web; and
drying the fibrous web.

15. The method of claim 14, wherein the bacteria belonging to the family Thermicanaceae is determined from a sample obtained from the cellulose fiber suspension, process water for a production method of a fibrous web, the fibrous web, or the machine for producing a fibrous web by a molecular method, a nucleic acid-based method or by utilizing one or more primers and/or probes.

16. The method of claim 14, wherein the bacteria belonging to the family Thermicanaceae is controlled by decreasing a level of said bacteria at least by 5%.

17. The method of claim 14, wherein at least one of the one or more biocides is a non-oxidizing biocide selected from the group consisting of: 2,2-Dibromo3-nitrilopropionamide (DBNPA); 2-Bromo-2-nitropropane-1,3-diol (Bronopol); 2-Bromo-2-nitro-propan-1-ol (BNP); 2,2-Dibromo-2-cyano-N-(3-hydroxypropyl)acetamide; 2,2-Dibromomalonamide; 1,2-Dibromo-2,4-dicyanobutane (DCB); Bis(trichloromethyl)sulfone; 2-Bromo-2-nitrostyrene (BNS); Didecyl-dimethylammonium chlorine (DDAC); ADBAC and other quaternary ammonium compounds; 3-Iodopropynyl-N-butylcarbamate (IPBC); Methyl and Dimethyl-thiocarbamates and their salts; 5-Chloro-2-methyl-4-isothiazolin-3-one (CMIT); 2-Methyl-4-isothiazolin-3-one (MIT) and their mixture; 2-n-Octyl-4-isothiazolin-3-one (OIT); 4,5-Dichloro-2-(n-octyl)-3(2H)-isothiazolone (DCOIT); 4,5-Dichloro-1,2-dithiol-3-one; 1,2-Benzisothiazolin-3-one (BIT); 2-(Thiocyanomethylthio)benzthiazole (TCMBT); 2-Methyl-1,2-benzisothiazolin-3(2H)-one (MBIT); Tetrakis hydroxymethyl phosphonium sulfate (THPS); Tetrahydro-3,5-dimethyl-2H-1,3,5-thiadiazine-2-thione (Dazomet); Methylene bisthiocyanate (MBT); Ortho-phenylphenol (OPP) and its salts; Glutaraldehyde; Ortho-phthaldehyde (OPA); Guanidines and biguanidines; N-dodecylamine or n-dodecylguanidine; dodecylamine salt or dodecylguanidine salt, such as dodecylguanidine hydrochloride; Bis-(3-aminopropyl)dodecylamine; Pyrithiones, such as Zinc pyrithione; Triazines such as Hexahydro-1,3,5-trimethyl-1,3,5-triazine; 3-[(4-Methylphenyl)sulfonyl]-2-propenenitrile; 3-Phenylsulphonyl-2-propenenitrile; 3-[(4-trifluormethylphenyl)sulphonyl]-2-propenenitrile; 3-[(2,4,6-trimethylphenyl)sulphonyl]-2-propenenitrile; 3-(4-methoxyphenyl)sulphonyl-2-propenenitrile; 3-[(4-methylphenyl)sulphonyl]prop-2-enamide; any of their isomers; and any combination thereof; and/or
at least one of the one or more biocides is an oxidizing biocide selected from the group consisting of: chlorine; alkali and alkaline earth hypochlorite salts; hypochlorous acid; bromine; alkali and alkaline earth hypobromite salts; hypobromous acid; chlorine dioxide; ozone; hydrogen peroxide; peroxy compounds, such as performic acid, peracetic acid, percarbonate or persulfate; halogenated hydantoins, such as monohalodimethylhydantoins; dihalodimethylhydantoins; perhalogenated hydantoins; monochloramines; monobromamines; dihaloamines; trihaloamines; urea reacted with an oxidant, the oxidant being e.g. alkali and alkaline earth hypochlorite salts or alkali and alkaline earth hypobromite salts; ammonium salts, e.g. ammonium bromide, ammonium sulfate or ammonium carbamate, reacted with an oxidant, the oxidant being preferably alkali and alkaline earth hypochlorite salts or alkali and alkaline earth hypobromite salts; and any combination thereof.

18. A Thermicanaceae-specific primer, probe, or primer pair capable of specific hybridization with an rRNA gene or an rRNA polynucleotide thereof of a bacterium belonging to the family Thermicanaceae for determining said bacterium in a sample.

19. (canceled)

20. A Thermicanaceae-specific kit for determining a bacterium belonging to the family Thermicanaceae in a sample, wherein the kit comprises the primer, probe or primer pair of claim 18, and optionally reagents for determining said bacterium belonging to the family Thermicanaceae.

21. (canceled)

22. The kit of claim 20, wherein the kit is suitable for controlling bacteria belonging to the family Thermicanaceae in a cellulose fiber suspension or process water for a production method of a fibrous web, wherein the kit further comprises one or more biocides, and/or one or more enzyme inhibitors.


SEQUENCE LISTING (3 sequences)

SEQ ID NO: 1 - DNA, 1426 bp, Thermicanus aegyptius
cctggctcaggacgaacgctggcggcgtgcctaatacatgcaagtcggacggtttagaagtgagaggcttgcctctcgcttttagacagtggcggacgggtgagtaacacgtaggcaacctgcccgtaataccgggataacctggggaaaccgaggctaagcccggataggctccttttccgcatgggagaggagggaaagggtgcgacccgattacggaggggcctgcggcgcattagctagttggtgaggtaacggcccaccaaggcgatgatgcgtagccgacctgagagggtgaccggccacactgggactgagagacggcccagactcctacgggaggcagcagtagggaatcttcggcaatgggcgaaagcctgaccgagcaacgccgcgtgagtgaggaaggccttcgggttgtaaaactctgttgtttgggaagaagggaaagggtaggccccttaggtgacggtaccaaacgagaaagccccggctaactacgtgccagcagccgcggtaatacgtagggggcgagcgttgtccggaatgattgggcgtaaagcgcgcgcaggcggtcctttaagtctgatgtgaaagcccgcggcttaaccgcggaaggtcattggaaactgggggacttgaggctaggagagggaagtggaattcctggtgtagcggtgaaatgcgtagagatcaggaggaataccgatggcgaaagcagctttctggcctagaactgacgctgaggcgcgaaagcgtggggagcaaacaggattagataccctggtagtccacgccgtaaacgatgagtgctaggtgtgaggggtttcaaggcccttcgtgccgcagtacacacaataagcactccgcctggggagtacggtcgcaagtctgaaactcaaaggaattgacgggggcccgcacaagcggtggagcatgtggtttaattcgaagcaacgcgaagaaccttaccagggcttgacatccctctgacgggagtagagatacttcgtcccttcggggcagaggagaccaggtggtgcatggttgtcgtcagctcgtgtcgtgagatgttgggttaagtcccgcaacgagcgcaacccttgagcttagttgccagcattcagttgggcactctaggctgactgccgcagacgaagcggaagaaggtggggatgacgtcaaatcatcatgccccttatgtcctgggctacacacgtgctacaatggccggtacaaagggaagcgaaggaacgatccggaacggaacccaaaaagccggtcctagttcggattgcaggctgcaactcgcctgcatgaagtcggaatcgctagtaatcgcggatcagcatgccgcggtgaatccgttcccgggccttgtacacaccgcccgtcacaccacgagagtccgaaacacccgaagtcg

SEQ ID NO: 2 - DNA, 22 bp, Artificial Sequence
  A primer for Thermicanus aegyptius 16S rRNA gene
cttgaggctaggagagggaagt

SEQ ID NO: 3 - DNA, 21 bp, Artificial Sequence
  A primer for Thermicanus aegyptius 16S rRNA gene
caggcggagtgcttattgtgt